(19) United States
(12) Patent Application Publication
(10) Pub. No.: US 2010/0041661 A1
(43) Pub. Date: Feb. 18, 2010

(54) CASPASE INHIBITORS BASED ON PYRIDAZINONE SCAFFOLD

(75) Inventors: Hye Kyung Chang, Daejeon (KR); Yeong Soo Oh, Daejeon (KR); Yong Jin Jang, Daejeon (KR); Sung Sub Kim, Daejeon (KR); Kyeong Sik Min, Daejeon (KR); Chul Woong Chung, Daejeon (KR); Mi Jeong Park, Daejeon (KR); Jung Gyu Park, Daejeon (KR)

Correspondence Address: BIRCH STEWART KOLASCH & BIRCH, PO BOX 747, FALLS CHURCH, VA, 22040-0747, US

(73) Assignee: LG Life Sciences Ltd., Seoul (KR)

(21) Appl. No.: 12/514,244

(22) PCT Filed: Oct. 26, 2007
(86) PCT No.: PCT/KR2007/005303

§ 371 Date: Jun. 4, 2009

(30) Foreign Application Priority Data
Nov. 9, 2006 (KR) 10-2006-0110501

(51) Int. Cl. C07D 237/00

(52) U.S. Cl. 514/247; 544/239

(57) ABSTRACT

The present invention relates to a pyridazinone derivative which can be used as a caspase inhibitor, process for the preparation thereof, and pharmaceutical composition for inhibiting caspase comprising the same.

### TECHNICAL FIELD

The present invention relates to a pyridazinone derivative or pharmaceutically acceptable salt thereof as an inhibitor against various caspases including caspase-1 [interleukin-1β-converting enzyme, ICE], caspase-3 [apopain/CPP-32], caspase-8, and caspase-9, and a pharmaceutical composition for the inhibition of caspase comprising the same.

### BACKGROUND ART

Caspase is a new kind of cysteine protease in the form of α₂β₂ tetramer discovered during the last 10 years. About 14 kinds thereof have been known until now. Caspase-1(ICE), one of them, is a kind of cytokine and participates in converting the biologically inactive prointerleukin-1β to the active interleukin-1β. Interleukin-1 consists of interleukin-1α and interleukin-1β, both of which are synthesized in monocytes in the form of 31 KDa precursor. Only prointerleukin-1β is activated by ICE. The positions hydrolyzed by caspase-1 are Asp²⁷-Gly²⁸ and Asp¹¹⁶-Ala¹¹⁷. The hydrolysis of the latter position gives interleukin-1β. Interleukin-1β has been reported to act as an important mediator in causing inflammation (1,3). Caspase-1 has been discovered for the first time in 1989, and the three dimensional structure thereof was determined by X-ray crystallographic method by two independent study groups.

Caspase-3(CPP-32) is broadly studied for its role or mechanism for action, and its three dimensional structure was determined in 1996 (2). Caspase-3(apopain) activated from procaspase-3 is hydrolyzed at the position of (P₄)Asp-X-X-Asp(P₁) motif, and the known substrates include poly(ADP-ribose) polymerase, U1 70,000 Mr small nuclear ribonucleoprotein, catalytic subunit of 460,000 Mr DNA-dependent protein kinase, etc. The X-ray structure of caspase-7 has been reported to be very similar to that of caspase-3 (4).

Caspase-8 and 9 are present in the upstream of caspase-3,6,7, and all of these caspases are known to participate in the apoptosis cascade. The X-ray structure of caspase-8 was determined in 1999 (5), and particularly the inhibitors thereof may be advantageously used for treating the diseases related to apoptosis.

Caspase inhibitors mean these compounds that inhibit the activity of caspase, and so control such symptoms as inflammation, apoptosis, etc. caused by the caspase activity. Diseases or symptoms that may be treated or attenuated by administering the inhibitors include the following: dementia, cerebral stroke, brain impairment due to AIDS, diabetes, gastric ulcer, cerebral injury by hepatitis virus, hepatitis-induced hepatic diseases, acute hepatitis, fulminant hepatic failure, sepsis, organ transplantation rejection, rheumatic arthritis, ischemic cardiac diseases, and liver cirrhosis(6).

Among the caspase inhibitors known until now, the most noted irreversible inhibitors are the following:

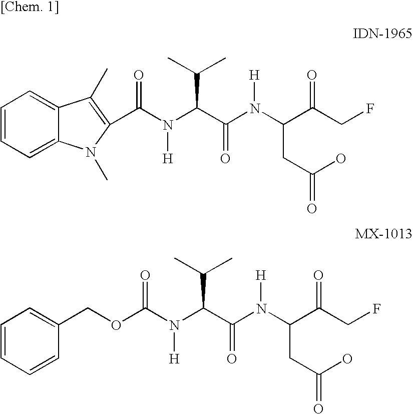

Both the above inhibitors exhibit their activity based on the common mechanism that they irreversibly inactivate the enzyme to suppress the cell apoptosis (irreversible, broad-spectrum inhibitor). It has been reported that irreversible inhibitor has much more effective inhibitory activity than reversible inhibitor (7). Both IDN-1965 of IDUN Co. and MX-1013 of Maxim Co. are reported to show activity in cell apoptosis model for hepatic injury (8, 9). These compounds are now in the stage of preclinical test.

The irreversible inhibitor IDN-6556 is now in the stage of phase II clinical trial as a hepatoprotective agent for hepatitis C patients (10, 6-liver cirrhosis-i).

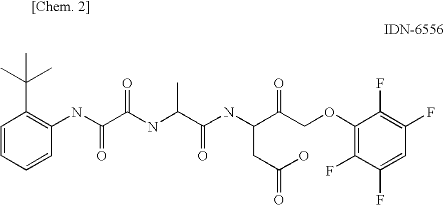

### REFERENCES

(1) Inflammation: Basic Principles and Clinical Correlates, 2nd ed., ed by Gallin, Goldstein and Snyderman. Raven Press Ltd., New York. 1992, pp. 211-232; Blood, 1996, 87(6), 2095-2147.
(2) Wilson, K. P. et al, Nature, 1994, 370. 270; Walker, N. P. C. et al. Cell, 1994, 78, 343; Nature Structural Biology, 1996, 3(7), 619.
(3) Thornberry, N. A. et al, Nature, 1992, 356. 768; Nature Biotechnology, 1996, 14, 297; Protein Science, 1995, 4, 3; Nature, 1995, 376(July 6), 37; Protein Science, 1995, 4, 2149.
(4) Wei, Y. et al, Chemistry and Biology, 2000, 7, 423.
(5) Blanchard H. et al, Structure, 1999, 7, 1125; Blanchard H. et al, J. of Mol. Biol., 2000, 302, 9.
(6) References for caspase related diseases
Dementia: Arch Neurol 2003 March; 60(3):369-76, Caspase gene expression in the brain as a function of the clinical progression of Alzheimer disease. Pompl P N, Yemul S, Xiang Z, Ho L, Haroutunian V, Purohit D, Mohs R, Pasinetti G M.
Cerebral stroke: Proc Natl Acad Sci USA 2002 Nov. 12; 99(23):15188-93, Caspase activation and neuroprotection in caspase-3-deficient mice after in vivo cerebral ischemia and in vitro oxygen glucose deprivation. Le D A, Wu Y, Huang Z, Matsushita K, Plesnila N, Augustinack J C, Hyman B T, Yuan J, Kuida K, Flavell R A, Moskowitz M A.
Brain impairment due to AIDS: J Neurosci 2002 May 15; 22(10):4015-24, Caspase cascades in human immunodeficiency virus-associated neurodegeneration. Garden G A, Budd S L, Tsai E, Hanson L, Kaul M, D'Emilia D M, Friedlander R M, Yuan J, Masliah E, Lipton S A.
Diabetes: Diabetes 2002 June; 51(6):1938-48, Hyperglycemia-induced apoptosis in mouse myocardium: mitochondrial cytochrome C-mediated caspase-3 activation pathway. Cai L, Li W, Wang G, Guo L, Jiang Y, Kang Y J.
Gastric ulcer: J Physiol Pharmacol 1998 December; 49(4):489-500, Role of basic fibroblast growth factor in the suppression of apoptotic caspase-3 during chronic gastric ulcer healing. Slomiany B L, Piotrowski J, Slomiany A.
Cerebral injury by hepatitis virus: J Viral Hepat 2003 March; 10(2):81-6, Cerebral dysfunction in chronic hepatitis C infection. Forton D M, Taylor-Robinson S D, Thomas H C.
Fulminant hepatic failure: Gastroenterology 2000 August; 119(2):446-60, Tumor necrosis factor alpha in the pathogenesis of human and murine fulminant hepatic failure. Streetz K, Leifeld L, Grundmann D, Ramakers J, Eckert K, Spengler U, Brenner D, Manns M, Trautwein C.
Sepsis: Nat Immunol 2000 December; 1(6):496-501, Caspase inhibitors improve survival in sepsis: a critical role of the lymphocyte. Hotchkiss R S, Chang K C, Swanson P E, Tinsley K W, Hui J J, Klender P, Xanthoudakis S, Roy S, Black C, Grimm E, Aspiotis R, Han Y, Nicholson D W, Karl I E.
Organ transplantation rejection: Xenotransplantation 2001 May; 8(2):115-24, In vitro prevention of cell-mediated xeno-graft rejection via the Fas/FasL-pathway in CrmA-transducted porcine kidney cells. Fujino M, Li X K, Suda T, Hashimoto M, Okabe K, Yaginuma H, Mikoshiba K, Guo L, Okuyama T, Enosawa S, Amemiya H, Amano T, Suzuki S.
Rheumatic arthritis: Prog Med Chem 2002; 39:1-72, Caspase inhibitors as anti-inflammatory and antiapoptotic agents. Graczyk P P.
Ischemic cardiac diseases: Am J Physiol Heart Circ Physiol 2002 September; 283(3):H990-5, Hypoxia-induced cleavage of caspase-3 and DFF45/ICAD in human failed cardiomyocytes. Todor A, Shafov V G, Tanhehco E J, Silverman N, Bernabei A, Sabbah H N.
Anti-inflammation: J Immunol 2003 Mar. 15; 170(6):3386-91, A broad-spectrum caspase inhibitor attenuates allergic airway inflammation in murine asthma model. Iwita A, Nishio K, Winn R K, Chi E Y, Henderson W R Jr, Harlan J M.
Hepatitis-induced hepatic diseases: i) J Viral Hepat. 2003 September; 10(5): 335-42. Apoptosis in hepatitis C Kountouras J, Zavos C, Chatzopoulos D.; ii) Apoptosis 2003 December; 8(6): 655-63 Apoptosis participates to liver damage in HSV-induced fulminant hepatitis. Pretet J L, Pelletier L, Bernard B, Coumes-Marquet S, Kantelip B, Mougin C.; iii) Proc Natl Acad Sci USA. 2003 Jun. 24; 100(13):7797-802. Caspase 8 small interfering RNA prevents acute liver failure in mice. Zender L, Hutker S, Liedtke C, Tillmann H L, Zender S, Mundt B, Waltemathe M, Gosling T, Flemming P, Malek N P, Trautwein C, Manns M P, Kuhnel F, Kubicka S.
Liver cirrhosis: i) J Pharmacol Exp Ther. 2004 March; 308(3): 1191-6, The caspase inhibitor Idn-6556 attenuates hepatic injury and fibrosis in the bile duct ligated mouse. Canbay A., Fledstein A., Baskin-Bey E., Bronk F. S. Gores G J.; ii) Hepatology. 2004 February; 39 (2): 273-8, Apoptosis: the nexus of liver injury and fibrosis. Canbay A, Friedman S, Gores G J.; iii) Hepatology. 2003 November; 38(5): 1188-98, Kupffer cell engulfment of apoptotic bodies stimulates death ligand and cytokine expression. Canbay A, Feldstein A E, Higuchi H, Werneburg N, Grambihler A, Bronk S F, Gores G J.
(7) Wu J. et al, Methods: A Companion to Methods in Enzymology, 1999, 17, 320.
(8) Hoglen N. C. et al, J. of Pharmacoloy and Experimental Therapeutics, 2001, 297, 811.
(9) Jaeschke H. et al, Toxicology and Applied Pharmacology, 2000, 169, 77.
(10) Hoglen N. C. et al, J. Pharmacol Exp. Ther., 2004, 309(2):634. Characterization of IDN-6556 (3-[2-(2-tert-butyl-phenylaminooxalyl)-amino]-propionylamino)-4-oxo-5-(2,3,5,6-tetrafluoro-phenoxy)-pentanoic acid): a liver-targeted caspase inhibitor.

### DISCLOSURE

### Technical Problem

The present inventors have extensively studied to design novel compounds which can be used as an effective and more selective inhibitor against caspases.

### Technical Solution

To achieve such a subject, the present inventors synthesized various compounds, and determined their binding ability and inhibitory activity for caspases. As a result, the inventors have discovered that a compound of the following formula (1) does meet such requirements, and completed the present invention.

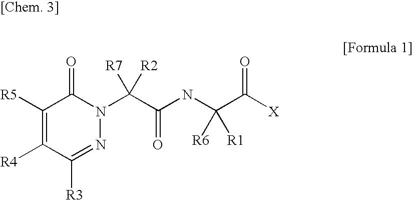

in which

R¹, R², R³, R⁴, R⁵, R⁶, R⁷ and X are defined below.

Therefore, the present invention provides the novel pyridazinone derivative of formula (1) or pharmaceutically acceptable salt thereof having effective inhibitory activity against caspases.

It is another object of the present invention to provide a pharmaceutical composition for inhibiting caspase, specifically a composition for preventing inflammation and apoptosis, comprising the compound of formula (1) or pharmaceutically acceptable salt thereof as an active ingredient together with the pharmaceutically acceptable carrier.

### ADVANTAGEOUS EFFECTS

The compound of formula (I) according to the present invention has an excellent inhibitory activity against caspase, and so can be advantageously used for the treatment of various diseases and symptoms mediated by caspase.

### BEST MODE

First of all, the important terms in the present invention are defined as follows:

a) C₁-C₅-alkyl: Straight-chain or branched hydrocarbons having 1 to 5 carbon atoms, that include methyl, ethyl, n-propyl, i-propyl, n-butyl, i-butyl, t-butyl, etc., but are not limited thereto.

b) C₃-C₁₀-cycloalkyl: Cyclic hydrocarbons having 3 to 10 carbon atoms, that include cyclopropyl, cyclobutyl, cyclopentyl, cyclohexyl, etc., but are not limited thereto.

c) Aryl: Aryl group includes all the aromatic, heteroaromatic and their partially reduced derivatives. The aromatic group means a 5 to 15-membered single or fused unsaturated hydrocarbon. The heteroaromatic group means the aromatic group containing 1 to 5 hetero atoms selected from a group consisting of oxygen, sulfur, and nitrogen. The aryl group includes phenyl, naphthyl, indolyl, quinolinyl, isoquinolyl, imidazolinyl, isoxazolyl, oxazolyl, thiazolyl, etc., but is not limited thereto.

One or more hydrogens in said C₁-C₅-alkyl, C₃-C₁₀-cycloalkyl or aryl group may be replaced with a group(s) selected from the following: acyl, amino, carboalkoxy, carboxy, carboxyamino, cyano, halo, hydroxy, nitro, thio, alkyl, cycloalkyl, alkoxy, aryl, aryloxy, sulfoxy, and guanido group.

d) Natural amino acid includes the following: Glycine, Alanine, Valine, Leucine, Isoleucine, Serine, Threonine, Cysteine, Methionine, Proline, Aspartic acid, Asparagine, Glutamic acid, Glutamine, Lysine, Arginine, Histidine, Phenylalanine, Tyrosine, and Tryptophan.

Further, the present specification includes the following abbreviations:

N-bromosuccinimide: NBS

O-(7-azabenzotriazol-1-yl)-N,N,N′,N′-tetramethyluroniumhexafluorophosphate]: HATU

N,N-dimethyl formamide: DMF

Dimethylsulfoxide: DMSO

N-methylmorpholine: NMM

2,2′-Azobis(2-methyl propionitrile): AIBN

2,2,6,6-Tetramethyl-1-piperidinyloxy, free radical: TEMPO

Lithium bis(trimethylsilyl)amide: LiHMDS

N-(2-Hydroxyethyl)piperazine-N′-(2′-ethanesulfonic acid): HEPES

3-[(3-Cholamidopropyl)dimethylamino]-1-propanesulfonate: CHAPS

Ethylenediaminetetraacetic acid: EDTA

Dithiothreitol: DTT

The present invention will be explained more in detail below. One aspect of the present invention relates to the pyridazinone derivative of the following formula (1):

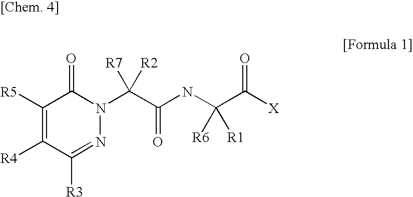

in which

I) R¹ represents H, C₁-C₅-alkyl, C₃-C₁₀-cycloalkyl, aryl, or a side chain residue of all the natural amino acids,

II) R² represents H, C₁-C₅-alkyl, C₃-C₁₀-cycloalkyl, aryl, or a side chain residue of all the natural amino acids,

III) R³ represents H, C₁-C₅-alkyl, aryl, hydroxy, C₃-C₁₀-alkoxy, or halogen,

IV) R⁴ represents H, C₁-C₅-alkyl, C₃-C₁₀-cycloalkyl, or aryl,

V) R⁵ represents H, C₁-C₅-alkyl, C₃-C₁₀-cycloalkyl, or aryl,

VI) R⁶ and R⁷ independently of one another each represent H, C₁-C₅-alkyl, C₃-C₁₀-cycloalkyl, or aryl,

VII) X represents —CH₂OR⁹ (R⁹ is C₁-C₅-alkyl, C₃-C₁₀-cycloalkyl, or aryl), —CH₂OC(═O)R¹⁰ (R¹⁰ is C₁-C₅-alkyl, C₃-C₁₀-cycloalkyl, or aryl), or —CH₂—W (W is halogen), or pharmaceutically acceptable salt thereof, which is useful as an inhibitor for caspase.

In the compound of formula (1) according to the present invention, R¹ preferably represents a side chain residue of all the natural amino acids, more preferably —CH₂ COOH. The compound of formula (1) may include the two kinds of stereoisomers, or mixtures thereof (diastereomeric mixtures) when the carbon to which R¹ is attached becomes a stereocenter due to the R¹ group. The compound of formula (1) may include an ester form (—CO Y wherein Y¹ is C₁-C₅-alkyl), a sulfonamide form (—CONHSO Y² wherein Y² is C₁-C₅-alkyl), and a pharmaceutically acceptable salt form, when R¹ is a side chain residue of an amino acid containing carboxyl moiety; or the compound of formula (1) may also exist in the form of a pharmaceutically acceptable salt when R¹ is a side chain residue of an amino acid containing a base moiety.

The compound of the present invention (formula 1a) may exist in the form of a cyclic ketal (formula 1b) when R¹ is —CH₂COOH, and so a skilled artisan may understand that the cyclic ketal form (formula 1b) may also be covered by the present invention.

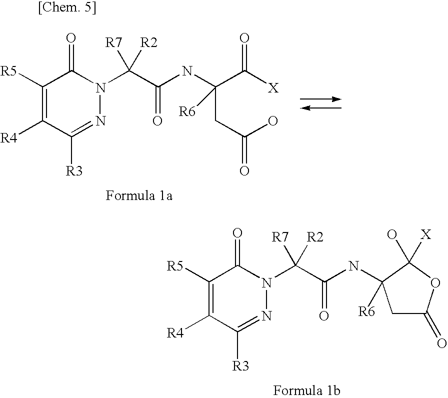

Also, the equilibrium forms of said compounds should be understood to cover their tautomeric forms.

R² preferably represents C₁-C₅-alkyl, more preferably methyl, ethyl, n-propyl, i-propyl, n-butyl, i-butyl, or t-butyl. The compound of formula (1) may include the two kinds of stereoisomers, or mixtures thereof (diastereomeric mixtures) when the carbon to which R² is attached becomes a stereocenter due to the R² group. The compound of formula (1) may include an ester form (—CO Y¹ wherein Y¹ is C₁-C₅-alkyl), a sulfonamide form (—CONHSO Y² wherein Y² is C₁-C₅-alkyl), and a pharmaceutically acceptable salt form, when R² is a side chain residue of an amino acid containing carboxyl moiety; or the compound of formula (1) may also exist in the form of a pharmaceutically acceptable salt when R² is a side chain residue of an amino acid containing a base moiety.

R³ preferably represents H, C₁-C₅-alkyl, aryl, C₁-C₅-alkoxy, or halogen, more preferably H, methyl, ethyl, n-propyl, i-propyl, n-butyl, i-butyl, or t-butyl, methoxy, ethoxy, fluoro, or chloro.

R⁴ preferably represents H.

R⁵ preferably represents C₁-C₅-alkyl substituted by C₃-C₁₀-cycloalkyl or aryl, each of which is substituted or unsubstituted; or represents substituted or unsubstituted aryl. R⁵ more preferably represents C₁-C₅-alkyl substituted by C₃-C₁₀-cycloalkyl or aryl, each of which is unsubstituted or substituted by one or more substituents selected from the group consisting of C₁-C₅-alkyl, hydroxy, C₁-C₅-alkoxy and halogen; or represents aryl which is unsubstituted or substituted by one or more substituents selected from the group consisting of C₁-C₅-alkyl, hydroxy, C₁-C₅-alkoxy and halogen. For example, R⁵ is phenyl, naphthyl, indolyl, quinolinyl, isoquinolyl, imidazolinyl, isoxazolyl, oxazolyl or thiazolyl, or is methyl substituted by phenyl, naphthyl, indolyl, quinolinyl, isoquinolyl, imidazolinyl, isoxazolyl, oxazolyl, thiazolyl or cyclohexyl, each of which is unsubstituted or substituted by one or more substituents selected from the group consisting of methyl, ethyl, n-propyl, i-propyl, n-butyl, i-butyl, t-butyl, methoxy, ethoxy, trihalomethyl and halogen.

R⁶ and R⁷ each preferably represent H.

R⁹ preferably represents aryl substituted by one or more halogens, more preferably phenyl substituted by one or more fluorines, and most preferably 2,3,5,6-tetrafluorophenyl.

R¹⁰ preferably represents aryl substituted by one or more halogens, more preferably phenyl substituted by one or more chlorines, most preferably 2,6-dichlorophenyl.

W preferably represents F.

The most preferred compounds are these selected from the following group:
3-{2-[5-(2-tert-butyl-benzyl)-3-methyl-6-oxo-6H-pyridazin-1-yl]-butyrylamino}-5-fluoro-4-oxo-pentanoic acid (1);
(S)-3-{2-[5-(2-tert-butyl-benzyl)-3-methyl-6-oxo-6H-pyridazin-1-yl]-butyrylamino}-4-oxo-5-(2,3,5,6-tetrafluoro-phenoxy)-pentanoic acid (2);
(S)-3-{2-[5-(2-tert-butyl-benzyl)-3-methyl-6-oxo-6H-pyridazin-1-yl]-propionylamino}-4-oxo-5-(2,3,5,6-tetrafluoro-phenoxy)-pentanoic acid (3);
(S)-3-{2-[5-(2-tert-butyl-benzyl)-3-methyl-6-oxo-6H-pyridazin-1-yl]-acetylamino}-4-oxo-5-(2,3,5,6-tetrafluoro-phenoxy)-pentanoic acid (4);
(S)-3-{2-[5-(2-tert-butyl-benzyl)-6-oxo-6H-pyridazin-1-yl]-butyrylamino}-4-oxo-5-(2,3,5,6-tetrafluoro-phenoxy)-pentanoic acid (5); and
(S)-3-{2-[3-(2-tert-butyl-benzyl)-6-oxo-6H-pyridazin-1-yl]-butyrylamino}-4-oxo-5-(2,3,5,6-tetrafluorophenoxy)-pentanoic acid (6).

The processes for preparation of the novel pyridazinone derivative of formula (1) showing an inhibitory activity against caspases are depicted in the following Reaction Schemes 1 to 3. However, these illustrated in the following Reaction Schemes represent only the typical processes used in the present invention. The manipulation order, reagent, reaction condition, solvent, etc. may be changed with no limit.

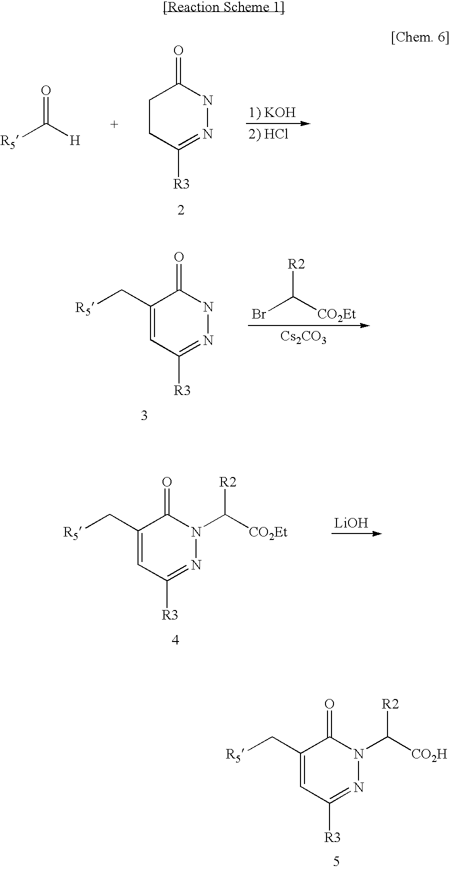

In the above Reaction Scheme, R5′ represents R5 except for CH₂ group.

In Reaction Scheme 1, the aromatic aldehyde and 6-alkyl-4,5-dihydro-2H-pyridazin-3-one are reacted in ethanol in the presence of a base to give the pyridazinone compound (3). This compound (3) is reacted with α-halo-α-alkylacetate in a suitable solvent in the presence of a base to give the compound (4). If necessary, the compound (4) is hydrolyzed to give the deprotected carboxylic acid derivative (5).

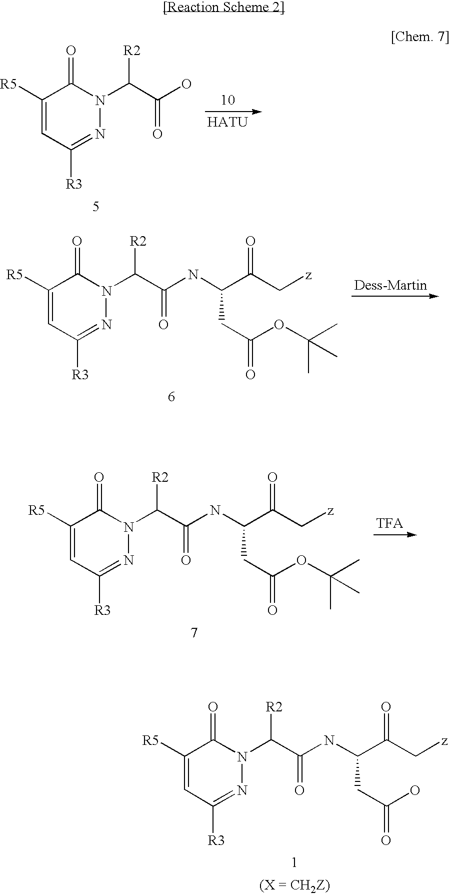

In the above Reaction Scheme 2 and the following Reaction Scheme 3, Z represents —OR⁹ (R⁹ is C₁-C₅-alkyl, C₃-C₁₀-cycloalkyl, or aryl), —OC(═O)R¹⁰ (R¹⁰ is C₁-C₅-alkyl, C₃-C₁₀-cycloalkyl, or aryl), or —W (W is halogen).

As depicted in Reaction Scheme 2, the carboxylic acid derivative (5) is coupled with the aspartic acid derivative (10) (see the following Reaction Scheme 3) to give the compound (6), which is then subjected to Dess-Martin periodene oxidation reaction, and if necessary deprotection reaction, to give the desired compound (1).

The functional group Z in the compound (1) of Reaction Scheme 2 may be formed first by synthesizing the compound (10) already having the desired Z group according to the process of Reaction Scheme 3, and by reacting the compound (10) with the carboxylic acid compound (5) (see WO 00/23421). Or, the desired Z group may be introduced later according to the process of Reaction Scheme 3 after the carboxylic acid compound (5) is combined with the aspartic acid (P-t-Bu) methyl ester and hydrolyzed. When Z is F, the racemic compound may be prepared according to a method known in Tetrahedron Letters, 1994, 35(52), 9693-9696.

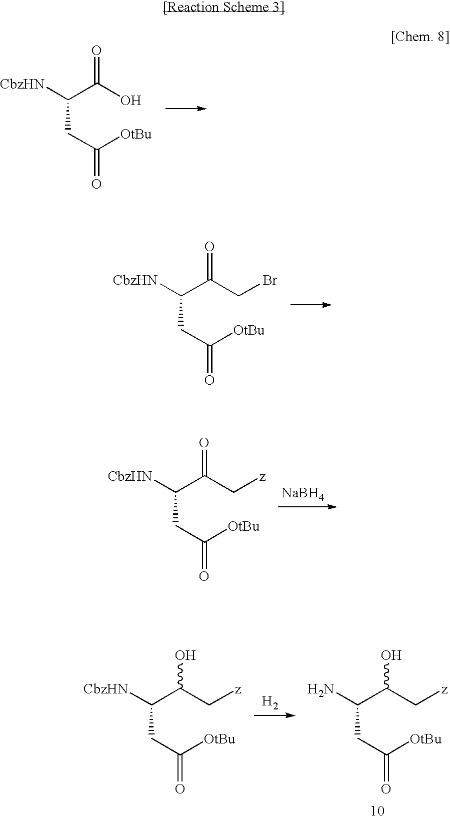

The compound of formula (1) according to the present invention has a broad spectrum of inhibitory activity against caspases as demonstrated by the results of the following Experiments, and so has an effect for preventing inflammation and apoptosis. Thus, the present invention provides a pharmaceutical composition for inhibiting caspases, specifically a therapeutic composition for preventing inflammation and apoptosis, comprising the compound of formula (1) or pharmaceutically acceptable salt thereof as an active ingredient together with the pharmaceutically acceptable carrier. Specifically, the composition of the present invention has a therapeutic or preventing effect for dementia, cerebral stroke, brain impairment due to AIDS, diabetes, gastric ulcer, cerebral injury by hepatitis, hepatitis-induced hepatic diseases, acute hepatitis, fulminant hepatic failure, sepsis, organ transplantation rejection, rheumatic arthritis, cardiac cell apoptosis due to ischemic cardiac diseases, or liver cirrhosis.

Further, the present invention provides a use of the compound of formula (1) or pharmaceutically acceptable salt thereof for inhibiting caspase, specifically for preventing inflammation and apoptosis. The present invention still further provides a method for preventing inflammation and apoptosis in a patient, which comprises administering a therapeutically effective amount of the compound of formula (1) or pharmaceutically acceptable salt thereof to the patient. The present invention still further provides a method for the treatment or prevention of dementia, cerebral stroke, brain impairment due to AIDS, diabetes, gastric ulcer, cerebral injury by hepatitis, hepatitis-induced hepatic diseases, acute hepatitis, fulminant hepatic failure, sepsis, organ transplantation rejection, rheumatic arthritis, cardiac cell apoptosis due to ischemic cardiac diseases, or liver cirrhosis in a patient, which comprises administering a therapeutically effective amount of the compound of formula (1) or pharmaceutically acceptable salt thereof to the patient.

The compound of formula (1) may be formulated into various pharmaceutical forms for administration purpose. To prepare the pharmaceutical composition according to the present invention, an effective amount of the compound of formula (1) or pharmaceutically acceptable salt thereof is mixed with a pharmaceutically acceptable carrier that may be selected depending on the formulation to be prepared.

The caspase inhibitor compound may be formulated as a parenteral injection, percutaneous or oral preparation, depending on its application purpose. It is especially advantageous to formulate the composition in a unit dosage form for ease of administration and uniformity of dosage.

For the oral preparation, any usual pharmaceutical carrier may be used. For example, water, glycols, oils, alcohols and the like may be used for such oral liquid preparations as suspensions, syrups, elixirs and solutions; or starches, sugars, kaolin, lubricants, binders, disintegrating agents and the like may be used for such solid preparations as powders, pills, capsules and tablets. Due to their ease of administration, tablets and capsules are the most advantageous dosage unit forms. It is also desirable for tablets and pills to be formulated into enteric-coated preparation.

For the parenteral preparation, sterile water is usually used as the carrier, though other ingredients such as solubility aids may be used. Injections, for example, sterilized aqueous or oily suspension for injection, can be prepared according to the known procedure using suitable dispersing agent, wetting agent, or suspending agent. Solvents that can be used for preparing injections include water, Ringer's fluid, and isotonic NaCl solution, and also sterilized fixing oil may be conveniently used as the solvent or suspending media. Any non-stimulative fixing oil including mono- or di-glyceride may be used for this purpose. Fatty acid such as oleic acid may also be used for injections.

For the percutaneous administration, the carrier may include a penetration enhancing agent and/or a suitable wetting agent, optionally combined with suitable additives having no significant skin irritation. Said additives may facilitate the administration through the skin and/or may assist preparation of a desired composition. These percutaneous preparations are administered via various manners, e.g., as a transdermal patch, a spot-on, or an ointment.

When the caspase inhibitor of the present invention is used for clinical purpose, it is preferable to administer to the subject patient in an amount ranging from 0.1 to 100 mg per kg of body weight a day. The total daily dosage may be administered once or over several times. However, specific administration dosage for an individual patient can be varied with specific compound used, body weight, gender, hygienic condition, or diet of subject patient, time or method of administration, excretion rate, mixing ratio of agent, severity of disease to be treated, etc.

### MODE FOR INVENTION

The present invention will be more specifically explained by the following examples. However, it should be understood that these examples are intended to illustrate the present invention but not in any manner to limit the scope of the present invention.

### Preparation 1-1

### 1-Bromomethyl-2-tert-butyl-benzene

To 1-tert-butyl-2-methyl-benzene (940 mg, 6.34 mmol), NBS (1.24 g, 1.1 eq) and AIBN (20 mg, catalytic amount) was added CCl₄ (12 ml), and the mixture was refluxed for 1 h. The suspended particles were removed by filtration, and washed with CCl₄. The organic layers were combined, and concentrated under reduced pressure to give 1.5 g of a yellow liquid in a stoichiometric yield.

¹H-NMR (500 MHz, CDCl₃) δ 7.46 (m, 1H), 7.38 (m, 1H), 7.22-7.21 (m, 2H), 4.83 (s, 2H), 1.46 (s, 9H)

### Preparation 1-2

### 2-tert-Butyl-benzaldehyde

To the compound of Preparation 1-1) (1.00 g, 4.4 mmol) were added NaHCO₃ (1.85 g, 5.0 eq) and DMSO (10 ml), and the mixture was heated at 100° C. for 30 min. The reaction mixture was extracted with ethyl acetate (100 ml×2), washed with water (50 ml×3) and aqueous sodium chloride solution (50 ml×1), dried (anhydrous Na₂SO₄), and concentrated under reduced pressure. The residue was purified by column chromatography (5% ethyl acetate-hexane) to give the title compound (750 mg, Yield 99%).

¹H-NMR (500 MHz, CDCl₃) δ 10.85 (s, 1H), 7.93 (d, 1H), 7.49 (m, 1H), 7.32 (m, 1H), 7.25 (m, 1H), 1.52 (s, 9H)

### Preparation 1-3

### 4-(2-tert-Butyl-benzyl)-6-methyl-2H-pyridazin-3-one

To the compound of Preparation 1-2) (324 mg, 2.0 mmol) were added 6-methyl-4,5-dihydro-2H-pyridazin-3-one (Aldrich, 224 mg, 1.0 eq), KOH (168 mg, 3.0 eq) and EtOH (10 ml), and the mixture was heated under reflux for 18 h. The reaction mixture was neutralized by 1N aqueous hydrochloric acid solution (3.0 ml), and distilled under reduced pressure. The residue was dissolved in excess ethyl acetate (50 ml), washed with aqueous sodium chloride solution, dried (anhydrous Na₂SO₄), and concentrated under reduced pressure. The residue was purified by column chromatography (50% ethyl acetate-hexane) to give the title compound (292 mg, Yield 57%).

¹H-NMR (500 MHz, CDCl₃) δ 12.66 (br s, 1H), 7.48 (d, 1H), 7.26-7.20 (m, 2H), 7.02 (d, 1H), 6.40 (s, 1H), 4.22 (s, 2H), 2.19 (s, 3H), 1.34 (s, 9H)

### Preparation 1-4

### 2-[5-(2-tert-Butyl-benzyl)-3-methyl-6-oxo-6H-pyridazin-1-yl]-butyric acid ethyl ester

To a mixture of the compound of Preparation 1-3) (90 mg, 0.35 mmol) and Cs₂CO₃ (342 mg, 3.0 eq) were added DMF (7 ml) and 2-bromo-butyric acid ethyl ester (343 mg, 5.0 eq), and the mixture was stirred under nitrogen gas at room temperature for 3 h. The reaction mixture was concentrated under reduced pressure, and the residue was extracted twice with ethyl acetate (100 ml). The extract was washed with saturated sodium hydrogen carbonate solution (NaHCO₃, 100 ml×2) and aqueous sodium chloride solution, dried (anhydrous Na₂SO₄), and concentrated under reduced pressure. The residue was purified by column chromatography (20% ethyl acetate-hexane) to give the title compound in a stoichiometric yield.

¹H-NMR (500 MHz, CDCl₃) δ 7.46 (d, 1H), 7.25-7.18 (m, 2H), 7.02 (d, 1H), 6.33 (s, 1H), 5.43 (m, 1H), 4.19 (m, 1H), 4.17 (s, 2H), 2.24 (m, 2H), 2.16 (s, 3H), 1.33 (s, 9H), 1.22 (t, 3H), 0.91 (t, 3H)

### Preparation 1-5

### 2-[5-(2-tert-Butyl-benzyl)-3-methyl-6-oxo-6H-pyridazin-1-yl]-butyric acid

The compound of Preparation 1-4) (128 mg) was dissolved in a solvent mixture (6 ml, tetrahydrofuran:MeOH:H₂O=3:2:1), LiOH.H₂O (29 mg, 2.0 eq) was added thereto, and the mixture was stirred at room temperature for about 2 h. The reaction mixture was neutralized by 1N aqueous hydrochloric acid solution, and distilled under reduced pressure to remove most tetrahydrofuran. The residue was dissolved in excess ethyl acetate (50 ml), washed with aqueous sodium chloride solution, dried (anhydrous Na₂ SO₄), and concentrated under reduced pressure to give the title compound in a stoichiometric yield. This compound was used in the next reaction without further purification.

### Preparation 1-6

### 3-{2-[5-(2-tert-Butyl-benzyl)-3-methyl-6-oxo-6H-pyridazin-1-yl]-butyrylamino}-5-fluoro-4-oxo-pentanoic acid tert-butyl ester

A mixture of the carboxylic acid derivative obtained in Preparation 1-5) (125 mg, 0.36 mmol), 3-amino-5-fluoro-4-hydroxy-pentanoic acid tert-butyl ester (see: Tetrahedron Letters, 1994, 35(52), 9693-9696, 83 mg, 1.1 eq) and HATU (178 mg, 1.3 eq) was cooled to 0° C., triethylamine (0.20 ml, 4.0 eq) in DMF solvent (5 ml) was added thereto, and the mixture was reacted at room temperature for 3 h. The solvent was distilled under reduced pressure. The residue was extracted with ethyl acetate (30 ml×2), washed with water, aqueous sodium hydrogen carbonate solution and aqueous sodium chloride solution, dried (anhydrous Na₂SO₄), and concentrated under reduced pressure. To the compound thus obtained and Des s-Martin reagent (305 mg, 2.0 eq) was added anhydrous dichloromethane (4 ml), and the mixture was stirred at room temperature for 1 h. Isopropyl alcohol (1 ml) was added to stop the reaction. The reaction mixture was filtered through celite under reduced pressure to remove the solid, and extracted with ethyl acetate (20 ml×2). The extract was washed with water, saturated sodium hydrogen carbonate solution and aqueous sodium chloride solution, dried (anhydrous Na₂SO₄), and concentrated under reduced pressure. The residue was purified by Prep-TLC (30-40% ethyl acetate-hexane) to give the title compound (125 mg, Yield 67%).

¹H-NMR (500 MHz, CDCl₃) δ 7.48-7.42 (m, 2H), 7.24 (t, 1H), 7.18 (t, 1H), 6.99 (m, 1H), 6.36 (s, 1H), 5.49 (m, 1H), 5.18-4.90 (m, 2H), 4.83 (m, 1H), 4.17 (s, 2H), 2.98-2.62 (m, 2H), 2.19 (two s, 3H), 2.25-2.12 (m, 2H), 1.39 (two s, 9H), 1.32 (s, 9H), 0.90 (m, 3H)

### Example 1

### 3-{2-[5-(2-tert-Butyl-benzyl)-3-methyl-6-oxo-6H-pyridazin-1-yl]-butyrylamino}-5-fluoro-4-oxo-pentanoic acid

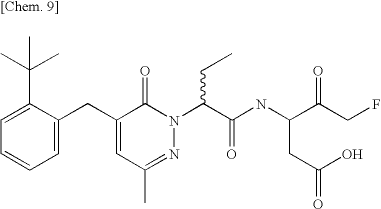

The compound of Preparation 1-6) (120 mg, 0.23 mmol) was dissolved in dichloromethane (4 ml), and trifluoroacetic acid (2 ml) was added thereto at 0° C. The reaction mixture was stirred for 1 h while being slowly warmed to room temperature, and concentrated under reduced pressure. The residue was purified by Prep-TLC (10% methanol-dichloromethane) to give the title compound (90 mg, Yield 82%).

¹H-NMR (500 MHz, CDCl₃) δ 7.70 (two br s, 1H), 7.46 (d, 1H), 7.24 (t, 1H), 7.18 (t, 1H), 6.97 (d, 1H), 6.46 & 6.43 (two s, 1H), 5.37 (m, 1H), 5.05-4.70 (m, 3H), 4.14 (s, 2H), 3.18-2.72 (m, 2H), 2.23 (two s, 3H), 2.25-2.15 (m, 2H), 1.32 (s, 9H), 0.92 (m, 3H)

### Preparation 2-1

### (S)-3-Benzyloxycarbonylamino-4-hydroxy-5-(2,3,5,6-tetrafluoro-phenoxy)-pentanoic acid tert-butyl ester

To N-benzyloxycarbonyl-β-t-butylaspartic acid (17.93 g, 55.46 mmol) and NMM (6.70 ml, 1.10 eq) was added anhydrous tetrahydrofuran (150 ml) under nitrogen gas, which was maintained at −15° C. Isobutylchloroformate (7.56 ml, 1.05 eq) was added thereto, and reaction mixture was stirred for about 20 min. The mixture was maintained at 0° C., during which diazomethane-ether solution (synthesized from 2.0 eq 1-methyl-3-nitro-1-nitroso-guanidine, 60 ml) was added, and stirred at 0° C. for 30 min to give a diazoketone derivative. 30% HBr/AcOH (22.6 ml, 2.0 eq) was added thereto at 0° C., and stirred for 30 min. The reaction mixture was extracted with ethyl acetate, washed with water, saturated sodium hydrogen carbonate solution (twice) and aqueous sodium chloride solution, dried (anhydrous Na₂SO₄), and concentrated under reduced pressure to give a bromomethylketone derivative (22.2 g) in a stoichiometric yield.

The bromomethylketone derivative (22.2 g, 55.45 mmol) and 2,3,5,6-tetrafluorophenol (11.05 g, 1.2 eq) were dissolved in dimethylformamide (130 ml), KF (8.05 g, 2.5 eq) was added, and the mixture was stirred at room temperature for 2 h. The reaction mixture was concentrated under reduced pressure. The residue was extracted with ethyl acetate, washed with water, saturated sodium hydrogen carbonate solution (twice) and aqueous sodium chloride solution, dried (anhydrous Na₂SO₄), and concentrated under reduced pressure to give 2,3,5,6-tetrafluorophenoxymethylketone derivative. This compound was dissolved in methanol (150 ml), NaBH₄ (4.19 g, 2.0 eq) was slowly added thereto at 0° C., and the mixture was stirred for 1 h. Saturated ammonium acetate solution was added to stop the reaction, and the reaction mixture was distilled under reduced pressure to remove methanol. The residue was extracted with ethyl acetate (200 ml×2), washed with water and aqueous sodium chloride solution, dried (anhydrous Na₂SO₄), and concentrated under reduced pressure. The residue was purified-separated by column chromatography (10-20% ethyl acetate/hexane) to give the title compound (19.6 g, Yield 73%).

### Preparation 2-2

### (S)-3-Amino-4-hydroxy-5-(2,3,5,6-tetrafluoro-phenoxy)-pentanoic acid tert-butyl ester

The compound of Preparation 2-1) (19.6 g, 40.2 mmol) was dissolved in MeOH (130 ml), Pd/C (Aldrich, 10%, 1.0 g) was added, and the mixture was stirred under hydrogen gas for 3 h. The reaction mixture was filtered through celite to remove Pd/C, and washed with MeOH. The filtrate was distilled under reduced pressure to give the title compound (13.17 g, Yield 93%).

¹H-NMR (400 MHz, DMSO-d₆) δ 8.2 (br, 2H), 7.6-7.5 (m, 1H), 5.9 (m, 1H), 4.3-4.1 (m, 3H), 3.6 (m, 1H), 2.7 (m, 1H), 1.4 (s, 9H)

### Preparation 2-3

### (S)-3-{2-[5-(2-tert-Butyl-benzyl)-3-methyl-6-oxo-6H-pyridazin-1-yl]-butyrylamino}-4-oxo-5-(2,3,5,6-tetrafluoro-phenoxy)-pentanoic acid tert-butyl ester

A mixture of the carboxylic acid derivative obtained in Preparation 1-5) (70 mg, 0.20 mmol), the compound of Preparation 2-2) (79 mg, 1.1 eq) and HATU (99 mg, 1.3 eq) was cooled to 0° C., triethylamine (0.11 ml, 4.0 eq) in DMF solvent (5 ml) was added thereto, and the mixture was reacted at room temperature for 1.5 h. The solvent was distilled under reduced pressure. The residue was extracted with ethyl acetate (30 ml×2), wished with water, aqueous sodium hydrogen carbonate solution and aqueous sodium chloride solution, dried (anhydrous Na₂SO₄), and concentrated under reduced pressure. To the compound thus obtained and Dess-Martin reagent (170 mg, 2.0 eq) was added anhydrous dichloromethane (4 ml), and the mixture was stirred at room temperature for 1 h. Isopropyl alcohol (1 ml) was added to stop the reaction. The reaction mixture was filtered through celite under reduced pressure to remove the solid, and extracted with ethyl acetate (20 ml×2). The extract was wished with water, saturated sodium hydrogen carbonate solution and aqueous sodium chloride solution, dried (anhydrous Na₂SO₄), and concentrated under reduced pressure. The residue was purified by Prep-TLC (30% ethyl acetate-hexane) to give the title compound (110 mg, Yield 81%).

¹H-NMR (500 MHz, CDCl₃) 7.54 (m, 1H), 7.47 (m, 1H), 7.19 (t, 1H), 7.00 (t, 1H), 6.75 (m, 1H), 6.37 (m, 1H), 5.50 (m, 1H), 5.16-4.96 (m, 2H), 4.86 (m, 1H), 4.17 (m, 2H), 3.03-2.61 (m, 2H), 2.20 (two s, 3H), 2.26-2.15 (m, 2H), 1.39 & 1.38 (two s, 9H), 1.33 (s, 9H), 0.91 (m, 3H)

### Example 2

### (S)-3-{2-[5-(2-tert-Butyl-benzyl)-3-methyl-6-oxo-6H-pyridazin-1-yl]-butyrylamino}-4-oxo-5-(2,3,5,6-tetrafluoro-phenoxy)-pentanoic acid

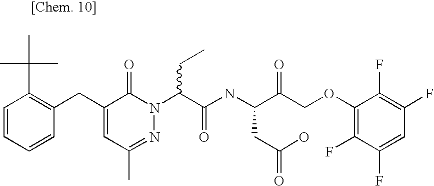

The compound of Preparation 2-3) (10 mg, 0.15 mmol) was dissolved in dichloromethane (4 ml), and trifluoroacetic acid (2 ml) was added thereto at 0° C. The reaction mixture was stirred for 1 h while being slowly warmed to room temperature, and concentrated under reduced pressure. The residue was purified by Prep-TLC (65% ethyl acetate-hexane) to give the title compound (85 mg, Yield 91%).

¹H-NMR (500 MHz, CDCl₃) δ 7.75 & 7.55 (two br s, 1H), 7.45 (m, 1H), 7.23 (t, 1H), 7.17 (m, 1H), 6.96 (m, 1H), 6.74 (m, 1H), 6.44 (two s, 1H), 5.43-5.34 (m, 1H), 5.00-4.70 (m, 3H), 4.12 (m, 2H), 3.11 (m, 1H), 2.77 (m, 1H), 2.20 & 2.21 (two s, 3H), 2.26-2.16 (m, 2H), 1.31 & 1.30 (two s, 9H), 0.92 (m, 3H)

### Preparation 3-1

### 2-[5-(2-tert-Butyl-benzyl)-3-methyl-6-oxo-6H-pyridazin-1-yl]-propionic acid ethyl ester

To a mixture of the compound of Preparation 1-3) (26 mg, 0.10 mmol) and Cs₂CO₃ (65 mg, 2.0 eq) were added DMF (5 ml) and 2-bromo-propionic acid ethyl ester (53 mg, 3.0 eq), and the mixture was stirred at room temperature under nitrogen gas for 1 h. The reaction mixture was concentrated under reduced pressure and the residue was extracted twice with ethyl acetate (100 ml). The extract was washed with saturated sodium hydrogen carbonate solution (NaHCO₃, 100 ml×2) and aqueous sodium chloride solution, dried (anhydrous Na₂SO₄), and concentrated under reduced pressure. The residue was purified by column chromatography (30% ethyl acetate-hexane) to give the title compound (30 mg, Yield 84%).

¹H-NMR (500 MHz, CDCl₃) δ 7.46 (d, 1H), 7.23 (t, 1H), 7.18 (t, 1H), 7.00 (d, 1H), 6.33 (s, 1H), 5.55 (qt, 1H), 4.20 (m, 2H), 4.16 (s, 2H), 2.16 (s, 3H), 1.68 (d, 3H), 1.33 (s, 9H), 1.23 (t, 3H)

### Preparation 3-2

### (S)-3-{2-[5-(2-tert-Butyl-benzyl)-3-methyl-6-oxo-6H-pyridazin-1-yl]-propionylamino}-4-oxo-5-(2,3,5,6-tetrafluoro-phenoxy)-pentanoic acid tert-butyl ester

The compound of Preparation 3-1) (30 mg, 0.084 mmol) was hydrolyzed according to the same procedure as Preparation 1-5) to give a carboxylic acid derivative (29 mg, 0.084 mmol). A mixture of this carboxylic acid derivative, the compound of Preparation 2-2) (35 mg, 1.1 eq) and HATU (44 mg, 1.3 eq) was cooled to 0° C., triethylamine (0.05 ml, 4.0 eq) in DMF solvent (5 ml) was added thereto, and the mixture was reacted at room temperature for 2 h. The solvent W is distilled under reduced pressure. The residue was extracted with ethyl acetate (30 ml×2), washed with water, aqueous sodium hydrogen carbonate solution and aqueous sodium chloride solution, dried (anhydrous Na₂SO₄), and concentrated under reduced pressure. To the compound thus obtained and Dess-Martin reagent (76 mg, 2.0 eq) was added anhydrous dichloromethane (4 ml), and the mixture was stirred at room temperature for 1 h. Isopropyl alcohol (1 ml) was added to stop the reaction. The reaction mixture was filtered through celite under reduced pressure to remove the solid, and extracted with ethyl acetate (20 ml×2). The extract was washed with water, saturated sodium hydrogen carbonate solution and aqueous sodium chloride solution, dried (anhydrous Na₂SO₄), and concentrated under reduced pressure. The residue was purified by Prep-TLC (40% ethyl acetate-hexane) to give the title compound (35 mg, Yield 60%).

¹H-NMR (500 MHz, CDCl₃) δ 7.47 (d, 1H), 7.37 (t, 1H), 7.24 (t, 1H), 7.18 (t, 1H), 6.99 (d, 1H), 6.73 (m, 1H), 6.37 (two s, 1H), 5.65 (m, 1H), 5.19-4.96 (m, 2H), 4.86 (m, 1H), 4.17 (s, 2H), 3.02-2.62 (m, 2H), 2.19 & 2.18 (two s, 3H), 1.68 (two d, 3H), 1.39 (s, 9H), 1.33 (s, 9H)

### Example 3

### (S)-3-{2-[5-(2-tert-Butyl-benzyl)-3-methyl-6-oxo-6H-pyridazin-1-yl]-propionylamino}-4-oxo-5-(2,3,5,6-tetrafluoro-phenoxy)-pentanoic acid

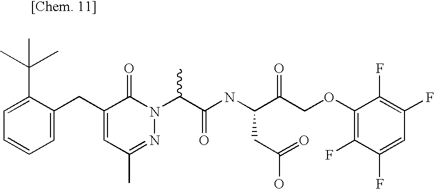

The compound of Preparation 3-2) (34 mg, 0.051 mmol) was dissolved in dichloromethane (4 ml), and trifluoroacetic acid (2 ml) was added thereto at 0° C. The reaction mixture was stirred for 1 h while being slowly warmed to room temperature, and concentrated under reduced pressure. The residue was purified by Prep-TLC (10% methanol/dichloromethane) to give the title compound (26 mg, Yield 84%).

¹H-NMR (500 MHz, CDCl₃) δ 7.61 (br, 1H), 7.46 (d, 1H), 7.24 (m, 1H), 7.18 (m, 1H), 6.95 (m, 1H), 6.76 (m, 1H), 6.45 (s, 1H), 5.51 (m, 1H), 4.89 (m, 3H), 4.12 (s, 2H), 3.14-2.73 (m, 2H), 2.21 (two s, 3H), 1.67 (two d, 3H), 1.31 (two s, 9H)

### Preparation 4-1

### [5-(2-tert-Butyl-benzyl)-3-methyl-6-oxo-6H-pyridazin-1-yl]-acetic acid ethyl ester

To a mixture of the compound of Preparation 1-3) (90 mg, 0.35 mmol) and Cs₂CO₃ (228 mg, 2.0 eq) were added DMF (10 ml) and 2-bromoacetic acid ethyl ester (117 mg, 2.0 eq), and the mixture was stirred at room temperature under nitrogen gas for 2 h. The reaction mixture was concentrated under reduced pressure and the residue was extracted twice with ethyl acetate (100 ml). The extract was washed with saturated sodium hydrogen carbonate solution (NaHCO₃, 100 ml×2) and aqueous sodium chloride solution, dried (anhydrous Na₂SO₄), and concentrated under reduced pressure. The residue was purified by column chromatography (30% ethyl acetate-hexane) to give the title compound (104 mg, Yield 87%).

¹H-NMR (500 MHz, CDCl₃) δ 7.46 (d, 1H), 7.23 (t, 1H), 7.19 (t, 1H), 7.01 (d, 1H), 6.35 (s, 1H), 4.87 (s, 2H), 4.24 (qt, 2H), 4.17 (s, 2H), 2.16 (s, 3H), 1.33 (s, 9H), 1.28 (t, 3H)

### Preparation 4-2

### (S)-3-{2-[5-(2-tert-Butyl-benzyl)-3-methyl-6-oxo-6H-pyridazin-1-yl]-acetylamino}-4-oxo-5-(2,3,5,6-tetrafluoro-phenoxy)-pentanoic acid tert-butyl ester

The compound of Preparation 4-1) (75 mg, 0.22 mmol) was hydrolyzed according to the same procedure as Preparation 1-5) to give a carboxylic acid derivative (60 mg, 0.19 mmol, 87%). A mixture of this carboxylic acid derivative, the compound of Preparation 2-2) (74 mg, 1.1 eq) and HATU (94 mg, 1.3 eq) was cooled to 0° C., triethylamine (0.11 ml, 4.0 eq) in DMF solvent (5 ml) was added thereto, and the mixture was reacted at room temperature for 2 h. The solvent was distilled under reduced pressure. The residue was extracted with ethyl acetate (30 ml×2), washed with water, aqueous sodium hydrogen carbonate solution and aqueous sodium chloride solution, dried (anhydrous Na₂SO₄), and concentrated under reduced pressure. To the compound thus obtained and Dess-Martin reagent (157 mg, 2.0 eq) was added anhydrous dichloromethane (4 ml), and the mixture was stirred at room temperature for 1 h. Isopropyl alcohol (1 ml) was added to stop the reaction. The reaction mixture was filtered through celite under reduced pressure to remove the solid, and extracted with ethyl acetate (20 ml×2). The extract was washed with water, saturated sodium hydrogen carbonate solution and aqueous sodium chloride solution, dried (anhydrous Na₂SO₄), and concentrated under reduced pressure. The residue was purified by Prep-TLC (40% ethyl acetate-hexane) to give the title compound (105 mg, Yield 80%).

¹H-NMR (500 MHz, CDCl₃) δ 7.47 (d, 1H), 7.32 (d, 1H), 7.25 (t, 1H), 7.19 (t, 1H), 6.99 (d, 1H), 6.74 (m, 1H), 6.40 (s, 1H), 5.24-5.03 (m, 2H), 4.91 (m, 1H), 4.85 (s, 2H), 4.16 (two s, 2H), 3.04-2.68 (m, 2H), 2.18 (s, 3H), 1.41 (s, 9H), 1.33 (s, 9H)

### Example 4

### (S)-3-{2-[5-(2-tert-Butyl-benzyl)-3-methyl-6-oxo-6H-pyridazin-1-yl]-acetylamino}-4-oxo-5-(2,3,5,6-tetrafluoro-phenoxy)-pentanoic acid

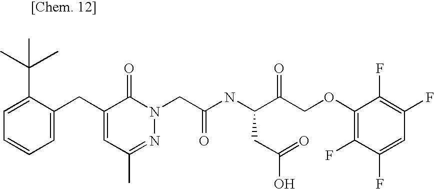

The compound of Preparation 4-2) (100 mg, 0.15 mmol) was dissolved in dichloromethane (4 ml), and trifluoroacetic acid (2 ml) was added thereto at 0° C. The reaction mixture was stirred for 1 h while being slowly warmed to room temperature, and concentrated under reduced pressure. The residue was purified by Prep-TLC (65% ethyl acetate/hexane) to give the title compound (59 mg, Yield 67%).

¹H-NMR (500 MHz, CDCl₃) δ 7.71 (br, 1H), 7.45 (d, 1H), 7.23 (t, 1H), 7.17 (t, 1H), 6.95 (d, 1H), 6.75 (m, 1H), 6.46 (s, 1H), 5.06-4.82 (m, 5H), 4.11 (s, 2H), 3.19-2.81 (m, 2H), 2.20 (s, 3H), 1.31 (s, 9H)

### Preparation 5-1

### 4-(2-tert-Butyl-benzyl)-2H-pyridazin-3-one and 6-(2-tert-Butyl-benzyl)-2H-pyridazin-3-one

To 4,5-dihydro-2H-pyridazin-3-one (192 mg, 1.95 mmol) obtained by a process known in J. Amer. Chem. Soc., 1945, 67, 60-62 and J. Org. Chem., 1961, 26, 1854-1856, 2-tert-butyl-benzaldehyde (316 mg, 1.0 eq) obtained in Preparation 1-2) and KOH (220 mg, 2.0 eq) was added EtOH (30 ml), and the mixture was heated under reflux for 6 h. The reaction mixture was neutralized by 1N aqueous hydrochloric acid solution, and distilled under reduced pressure to remove most tetrahydrofuran. The residue was dissolved in excess ethyl acetate (50 ml), washed with aqueous sodium chloride solution, dried (anhydrous Na₂SO₄), and concentrated under reduced pressure. The residue was purified by column chromatography (50% ethyl acetate-hexane, 10% methanol/dichloromethane) to give the title compounds 4-(2-tert-butyl-benzyl)-2H-pyridazin-3-one (76 mg) and 6-(2-tert-butyl-benzyl)-2H-pyridazin-3-one (167 mg).

### 4-(2-tert-Butyl-benzyl)-2H-pyridazin-3-one;

¹H-NMR (500 MHz, CDCl₃) δ 11.73 (s, 1H), 7.65 (d, 1H), 7.47 (d, 1H), 7.24 (t, 1H), 7.20 (t, 2H), 7.01 (d, 1H), 6.50 (m, 1H), 4.21 (s, 2H), 1.34 (s, 9H)

### 6-(2-tert-Butyl-benzyl)-2H-pyridazin-3-one;

¹H-NMR (500 MHz, CDCl₃) δ 10.60 (s, 1H), 7.61 (s, 1H), 7.45 (d, 1H), 7.25 (t, 1H), 7.18 (t, 2H), 6.97 (d, 1H), 6.44 (s, 1H), 4.15 (s, 2H), 1.40 (s, 9H)

### Preparation 5-2

### 2-[5-(2-tert-Butyl-benzyl)-6-oxo-6H-pyridazin-1-yl]-butyric acid ethyl ester

To a mixture of 4-(2-tert-butyl-benzyl)-2H-pyridazin-3-one obtained in Preparation 5-1) (76 mg, 0.314 mmol) and Cs₂CO₃ (307 mg, 3.0 eq) were added DMF (4 ml) and 2-bromobutyric acid ethyl ester (306 mg, 5.0 eq), and the mixture was stirred at room temperature under nitrogen gas for 2 h. The reaction mixture was concentrated under reduced pressure and the residue was extracted twice with ethyl acetate (100 ml). The extract was washed with saturated sodium hydrogen carbonate solution (NaHCO₃, 100 ml×2) and aqueous sodium chloride solution, dried (anhydrous Na₂SO₄), and concentrated under reduced pressure. The residue was purified by column chromatography (10-20% ethyl acetate-hexane) to give the title compound (100 mg, Yield 89%).

¹H-NMR (400 MHz, CDCl₃) δ 7.69 (d, 1H), 7.51 (d, 1H), 7.30-7.22 (m, 2H), 7.07 (d, 1H), 6.50 (m, 1H), 5.56 (dd, 1H), 4.25 (m, 4H), 2.35-2.21 (m, 2H), 1.38 (s, 9H), 1.28 (t, 3H), 0.98 (m, 3H)

### Preparation 5-3

### (S)-3-{2-[5-(2-tert-Butyl-benzyl)-6-oxo-6H-pyridazin-1-yl]-butyrylamino}-4-oxo-5-(2,3,5,6-tetrafluoro-phenoxy)-pentanoic acid tert-butyl ester

The compound of Preparation 5-2) (94 mg, 0.263 mmol) was hydrolyzed according to the same procedure as Preparation 1-5) to give a carboxylic acid derivative (86 mg, 0.263 mmol, 100%). A mixture of this carboxylic acid derivative, the compound of Preparation 2-2) (102 mg, 1.1 eq) and HATU (130 mg, 1.3 eq) was cooled to 0° C., triethylamine (0.15 ml, 4.0 eq) in DMF solvent (5 ml) was added thereto, and the mixture was reacted at room temperature for 2 h. The solvent W is distilled under reduced pressure. The residue was extracted with ethyl acetate (30 ml×2), washed with water, aqueous sodium hydrogen carbonate solution and aqueous sodium chloride solution, dried (anhydrous Na₂SO₄), and concentrated under reduced pressure. To the compound thus obtained and Dess-Martin reagent (223 mg, 2.0 eq) was added anhydrous dichloromethane (4 ml), and the mixture was stirred at room temperature for 1 h. Isopropyl alcohol (1 ml) was added to stop the reaction. The reaction mixture was filtered through celite under reduced pressure to remove the solid, and extracted with ethyl acetate (20 ml×2). The extract was washed with water, saturated sodium hydrogen carbonate solution and aqueous sodium chloride solution, dried (anhydrous Na₂SO₄), and concentrated under reduced pressure. The residue was purified by column chromatography (20-30% ethyl acetate-hexane) to give the title compound (150 mg, Yield 86%).

¹H-NMR (500 MHz, CDCl₃) δ 7.70 (m, 1H), 7.46 (d, 1H), 7.34 (m, 1H), 7.24 (t, 1H), 7.18 (t, 1H), 7.00 (m, 1H), 6.75 (m, 1H), 6.49 (m, 1H), 5.51 (m, 1H), 5.18-4.94 (m, 2H), 4.87 (m, 1H), 4.18 (m, 2H), 3.02-2.64 (m, 2H), 2.28-2.15 (m, 2H), 1.39 (two s, 9H), 1.32 (s, 9H), 0.92 (m, 3H)

### Example 5

### (S)-3-{2-[5-(2-tert-Butyl-benzyl)-6-oxo-6H-pyridazin-1-yl]-butyrylamino}-4-oxo-5-(2,3,5,6-tetrafluoro-phenoxy)-pentanoic acid

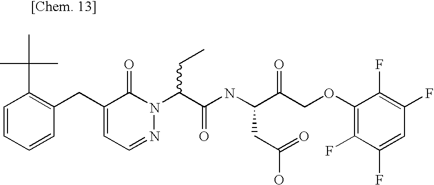

The compound of Preparation 5-3) (146 mg, 0.221 mmol) was dissolved in dichloromethane (4 ml), and trifluoroacetic acid (2 ml) was added thereto at 0° C. The reaction mixture was stirred for 1 h while being slowly warmed to room temperature, and concentrated under reduced pressure. The residue was purified by Prep-TLC (65% ethyl acetate/hexane) to give the title compound (116 mg, Yield 67%).

¹H-NMR (500 MHz, CDCl₃) 7.80 (m, 2H), 7.45 (d, 1H), 7.24 (m, 1H), 7.18 (m, 1H), 6.96 (m, 1H), 6.76 (m, 1H), 6.57 (m, 1H), 5.41-5.05 (m, 2H), 4.91 (m, 1H), 4.40 (m, 1H), 4.15 (s, 2H), 3.25-2.64 (m, 2H), 2.22 (m, 2H), 1.30 (two s, 9H), 0.94 (m, 3H)

### Preparation 6-1

### 2-[3-(2-tert-Butyl-benzyl)-6-oxo-6H-pyridazin-1-yl]-butyric acid ethyl ester

To a mixture of 6-(2-tert-butyl-benzyl)-2H-pyridazin-3-one obtained in Preparation 5-1) (167 mg, 0.689 mmol) and Cs₂CO₃ (673 mg, 3.0 eq) were added DMF (4 ml) and 2-bromobutyric acid ethyl ester (672 mg, 5.0 eq), and the mixture was stirred at room temperature under nitrogen gas for 2 h. The reaction mixture was concentrated under reduced pressure and the residue was extracted twice with ethyl acetate (100 ml). The extract was washed with saturated sodium hydrogen carbonate solution (NaHCO₃, 100 ml×2) and aqueous sodium chloride solution, dried (anhydrous Na₂SO₄), and concentrated under reduced pressure. The residue was purified by column chromatography (20% ethyl acetate-hexane) to give the title compound (189 mg, Yield 77%).

¹H-NMR (400 MHz, CDCl₃) δ 7.70 (d, 1H), 7.50 (d, 1H), 7.27 (t, 1H), 7.21 (t, 1H), 7.04 (d, 1H), 6.49 (d, 1H), 5.46 (dd, 1H), 4.25-4.19 (m, 4H), 2.31-2.15 (m, 2H), 1.43 (s, 9H), 1.27 (t, 3H), 0.93 (m, 3H)

### Preparation 6-2

### (S)-3-{2-[3-(2-tert-Butyl-benzyl)-6-oxo-6H-pyridazin-1-yl]-butyrylamino}-4-oxo-5-(2,3,5,6-tetrafluoro-phenoxy)-pentanoic acid tert-butyl ester

The compound of Preparation 6-1) (185 mg, 0.519 mmol) was hydrolyzed according to the same procedure as Preparation 1-5) to give a carboxylic acid derivative (166 mg, 98%). A mixture of this carboxylic acid derivative (87 mg, 0.263 mmol), the compound of Preparation 2-2) (102 mg, 1.1 eq) and HATU (130 mg, 1.3 eq) was cooled to 0° C., triethylamine (0.15 ml, 4.0 eq) in DMF solvent (5 ml) was added thereto, and the mixture was reacted at room temperature for 2 h. The solvent was distilled under reduced pressure. The residue was extracted with ethyl acetate (30 ml×2), washed with water, aqueous sodium hydrogen carbonate solution and aqueous sodium chloride solution, dried (anhydrous Na₂SO₄), and concentrated under reduced pressure. To the compound thus obtained and Dess-Martin reagent (223 mg, 2.0 eq) was added anhydrous dichloromethane (4 ml), and the mixture was stirred at room temperature for 1 h. Isopropyl alcohol (1 ml) was added to stop the reaction. The reaction mixture was filtered through celite under reduced pressure to remove the solid, and extracted with ethyl acetate (20 ml×2). The extract was washed with water, saturated sodium hydrogen carbonate solution and aqueous sodium chloride solution, dried (anhydrous Na₂SO₄), and concentrated under reduced pressure. The residue was purified by column chromatography (25-30% ethyl acetate-hexane) to give the title compound (150 mg, Yield 86%).

¹H-NMR (500 MHz, CDCl₃) δ 7.71 (d, 1H), 7.45 (d, 1H), 7.30 (t, 1H), 7.22 (t, 1H), 7.16 (m, 1H), 6.97 (d, 1H), 6.75 (m, 1H), 6.46 (d, 1H), 5.36 (m, 1H), 5.14-4.95 (m, 2H), 4.85 (m, 1H), 4.15 (m, 2H), 3.00-2.63 (m, 2H), 2.26-2.12 (m, 2H), 1.39 (three s, 18H), 0.90 (m, 3H)

### Example 6

### (S)-3-{2-[3-(2-tert-Butyl-benzyl)-6-oxo-6H-pyridazin-1-yl]-butyrylamino}-4-oxo-5-(2,3,5,6-tetrafluoro-phenoxy)-pentanoic acid

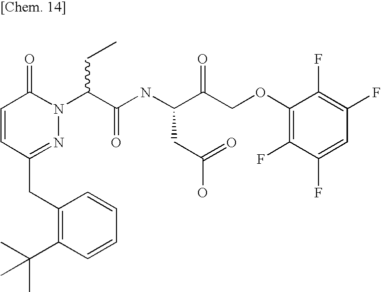

The compound of Preparation 6-2) (142 mg, 0.215 mmol) was dissolved in dichloromethane (4 ml), and trifluoroacetic acid (2 ml) was added thereto at 0° C. The reaction mixture was stirred for 1 h while being slowly warmed to room temperature, and concentrated under reduced pressure. The residue was purified by Prep-TLC (65% ethyl acetate/hexane) to give the title compound (111 mg, Yield 85%).

¹H-NMR (500 MHz, CDCl₃) δ 7.77 (d, 1H), 7.60 (br s, 1H), 7.45 (d, 1H), 7.22 (t, 1H), 7.16 (t, 1H), 6.95 (d, 1H), 6.76 (m, 1H), 6.51 (s, 1H), 5.28 (m, 1H), 5.05-4.40 (br s, 2H), 4.87 (m, 1H), 4.18 (m, 2H), 3.10-2.68 (m, 2H), 2.24-2.12 (m, 2H), 1.37 (two s, 18H), 0.91 (m, 3H)

Experiment 1

Assay for the Caspase Inhibitory Effect

Caspase-1 and caspase-8 known as cysteine proteases in the form of α₂β₂ were expressed, purified, and activated by modifying a method known in Thornberry, N. A. et al, Nature, 1992, 356, 768; Thornberry, N. A. Methods in Enzymology, 1994, 244, 615; Walker, N. P. C. et al. Cell, 1994, 78, 343, and caspase-9 was also purified by a similar method, and the inhibitory activity against them was tested. Briefly describing, p10 and p20 subunits (Thornberry, N. A. et al, Nature, 1992, 356, 768) were expressed in E. coli and purified by nickel column and anionic exchange chromatography to give caspase-1, caspase-8 and caspase-9. The fluorescent substrates AcYVAD-AFC for thus obtained caspase-1, AcDEVD-AFC for caspase-8, and AcLEHD-AFC for caspase-9, were used for determining specific activity of the synthesized inhibitors. The enzyme reaction was carried out at 25° C. with various concentrations of the inhibitors in a buffer solution containing 50 mM HEPES (pH 7.50), 10% (w/v) sucrose, 0.1% (w/v) CHAPS, 100 mM NaCl, 1 mM EDTA, and 10 mM DTT in the presence of 50 μM AcYVAD-AFC for 10 nM caspase-1, 50 μM AcDEVD-AFC for 2.1 nM caspase-8, and 150 μM AcLEHD-AFC for 200 nM caspase-9. The inhibitory constants Kᵢ and K_{obs} of the inhibitors were determined by measuring the reaction velocity with the time lapse using a fluorescent spectrometer and by obtaining the initial rate constant. Kᵢ was calculated from the Lineweaver Burk Plot, and K_{obs} from the following Equation 1.

K_{obs}=−ln(1−Aₜ/Aₒₒ)/t [Equation 1]

in which

Aₜ means cleavage rate (%) at time t, and

Aₒₒ means the maximum cleavage rate (%).

Spectra MAX GeminiXS Fluorescent Spectrometer of Molecular Device Co. was used at the excitation wavelength of 405 nm and the emission wavelength of 505 nm.

The in vivo inhibitory activity of the inhibitors was determined by subjecting Jurkat cell (ATCC TIB-152) to apoptosis using Fas antibody (Upstate Biotech 05-201) and by detecting the color change according to the WST-1 method known in Francoeur A. M. and Assalian A. (1996) Biochemica 3, 19-25 to observe the amount of alive Jurkat cells when the cells were treated by the inhibitor. Spectra MAX 340 Spectrometer of Molecular Device Co. was used at the absorbance wavelength of 440 nm.

TABLE 1

| | Caspase-8 | |
|---|---|---|
| | K_{obs}/[I] | Jurkat Cell |
| Example No. | (M⁻¹min⁻¹) | IC₅₀ (μM) |
| 1 | 5.5 E6 | 0.14 |
| 2 | 2.0 E6 | 0.33 |
| 3 | 4.0 E5 | |
| 4 | 2.0 E5 | |
| 5 | 2.0 E6 | 0.17 |
| 6 | 1.7 E5 | |

Experiment 2

Therapeutic Effect for Liver Injury Induced by Fas Antibody in Mouse

Step 1) Preparation of Blood Sample

Male Balb/c mice (6 weeks, Charles River Laboratory, Osaka, Japan) were kept under the conditions of 22° C., 55% of relative humidity, and light-darkness cycle of 12 hours. Food and water were supplied ad libitum. In pyrogen-free phosphate buffer was dissolved the Fas antibody (Jo2; BD pharmingen, San Diego, Calif.), which was then injected to each mouse in the amount of 0.15 mg/kg through the vein of tail. Immediately after the injection of the Fas antibody, vehicle (a mixture of PEG400:ethanol=2:1 was 20-fold diluted with phosphate buffer) wherein the test compound is dissolved or the vehicle alone was orally administered to the mice. After 6 hours from the drug administration, blood samples were obtained from their hearts.

Step 2: Assay for the Activity of Plasma Aminotransferase

The plasma ALT activity was determined for the blood samples obtained in Step 1 using ALT assay kit (Asan Pharm. Co., Seoul, Korea) according to the manufacturer's instruction. The results appeared that the injection of the Fas antibody sharply increases the ALT activity in plasma, and the test compounds inhibit the increased enzyme activity in a dose-dependent manner. Based on these results, ED₅₀ values of the test compounds were calculated using Prism software of GraphPad Co. to give 0.001-10 mg/kg.

### INDUSTRIAL APPLICABILITY

As the above results of Experiments show, the compound of formula (1) of the present invention has an excellent inhibitory activity against caspase, and particularly exhibits a therapeutic effect in the animal model of liver injury induced by the Fas antibody. Therefore, the compound of formula (1) can be advantageously used for the treatment or prevention of various diseases and symptoms mediated by caspase.

1. A compound of formula (1): 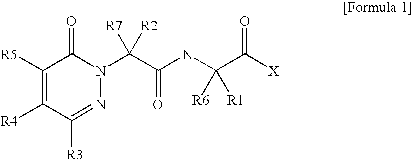
in which
I) R¹ represents H, C₁-C₅-alkyl, C₃-C₁₀-cycloalkyl, aryl, or a side chain residue of all the natural amino acids,
II) R² represents H, C₁-C₅-alkyl, C₃-C₁₀-cycloalkyl, aryl, or a side chain residue of all the natural amino acids,
III) R³ represents H, C₁-C₅-alkyl, aryl, hydroxy, C₁-C₅-alkoxy, or halogen,
IV) R⁴ represents H, C₁-C₅-alkyl, C₃-C₁₀-cycloalkyl, or aryl,
V) R⁵ represents H, C₁-C₅-alkyl, C₃-C₁₀-cycloalkyl, or aryl,
VI) R⁶ and R⁷ independently of one another each represent H, C₁-C₅-alkyl, C₃-C₁₀-cycloalkyl, or aryl,
VII) X represents —CH₂OR⁹ (R⁹ is C₁-C₅-alkyl, C₃-C₁₀-cycloalkyl, or aryl), —CH₂C(═O)R¹⁰ (R¹⁰ is C₁-C₅-alkyl, C₃-C₁₀-cycloalkyl, or aryl), or —CH₂—W (W is halogen), or pharmaceutically acceptable salt thereof.

2. The compound of claim 1 wherein R⁵ represents C₁-C₅-alkyl substituted by C₃-C₁₀-cycloalkyl or aryl, each of which is substituted or unsubstituted; or represents substituted or unsubstituted aryl, or pharmaceutically acceptable salt thereof.

3. The compound of claim 2 wherein R⁵ represents C₁-C₅-alkyl substituted by C₃-C₁₀-cycloalkyl or aryl, each of which is unsubstituted or substituted by one or more substituents selected from the group consisting of C₁-C₅-alkyl, hydroxy, C₁-C₅-alkoxy and halogen; or represents aryl which is unsubstituted or substituted by one or more substituents selected from the group consisting of C₁-C₅-alkyl, hydroxy, C₁-C₅-alkoxy and halogen, or pharmaceutically acceptable salt thereof.

4. The compound of claim 1 wherein
I) R¹ represents a side chain residue of all the natural amino acids,
II) R² represents C₁-C₅-alkyl,
III) R³ represents H, C₁-C₅-alkyl, aryl, C₁-C₅-alkoxy, or halogen,
IV) R⁴ represents H,
V) R⁵ represents C₁-C₅-alkyl substituted by C₃-C₁₀-cycloalkyl or aryl, each of which is unsubstituted or substituted by one or more substituents selected from the group consisting of C₁-C₅-alkyl, hydroxy, C₁-C₅-alkoxy and halogen; or represents aryl which is unsubstituted or substituted by one or more substituents selected from the group consisting of C₁-C₅-alkyl, hydroxy, C₁-C₅-alkoxy and halogen,
VI) R⁶ and R⁷ independently of one another each represent H,
VII) X represents —CH₂OR⁹ (R⁹ is C₁-C₅-alkyl, C₃-C₁₀-cycloalkyl, or aryl), —CH₂C(═O)R¹⁰ (R¹⁰ is C₁-C₅-alkyl, C₃-C₁₀-cycloalkyl, or aryl), or —CH₂—W (W is halogen), or pharmaceutically acceptable salt thereof.

5. The compound of claim 1 wherein
I) R¹ represents —CH₂COOH,
II) R² represents C₁-C₅-alkyl,
III) R³ represents H, C₁-C₅-alkyl, aryl, C₁-C₅-alkoxy, or halogen,
IV) R⁴ represents H,
V) R⁵ represents C₁-C₅-alkyl substituted by C₃-C₁₀-cycloalkyl or aryl, each of which is unsubstituted or substituted by one or more substituents selected from the group consisting of C₁-C₅-alkyl, hydroxy, C₁-C₅-alkoxy and halogen; or represents aryl which is unsubstituted or substituted by one or more substituents selected from the group consisting of C₁-C₅-alkyl, hydroxy, C₁-C₅-alkoxy and halogen,
VI) R⁶ and R⁷ independently of one another each represent H,
VII) X represents —CH₂O-(2,3,5,6-tetrafluorophenyl), —CH₂O-(2,6-dichlorobenzoyl) or —CH₂—F, or pharmaceutically acceptable salt thereof.

6. (S)-3-{2-[5-(2-tert-butyl-benzyl)-6-oxo-6H-pyridazin-1-yl]-butyrylamino}-4-oxo-5-(2,3,5,6-tetrafluoro-phenoxy)-pentanoic acid.

7. A pharmaceutical composition for inhibiting caspase, comprising the compound as defined in claim 1 or pharmaceutically acceptable salt thereof as an active ingredient together with a pharmaceutically acceptable carrier.

8. The composition of claim 7 for preventing inflammation and apoptosis.

9. The composition of claim 7 for the treatment or prevention of dementia, cerebral stroke, brain impairment due to AIDS, diabetes, gastric ulcer, cerebral injury by hepatitis, hepatitis-induced hepatic diseases, acute hepatitis, fulminant hepatic failure, sepsis, organ transplantation rejection, rheumatic arthritis, cardiac cell apoptosis due to ischemic cardiac diseases, or liver cirrhosis.

10. The composition of claim 7 for the treatment of acute hepatitis or liver cirrhosis.

11. The composition of claim 7 for the treatment of rheumatic arthritis.

12. A use of the compound as defined in claim 1 or pharmaceutically acceptable salt thereof for inhibiting caspase.

13. A method for preventing inflammation and apoptosis in a patient, which comprises administering a therapeutically effective amount of the compound as defined in claim 1 or pharmaceutically acceptable salt thereof to the patient.

14. A method for the treatment or prevention of dementia, cerebral stroke, brain impairment due to AIDS, diabetes, gastric ulcer, cerebral injury by hepatitis, hepatitis-induced hepatic diseases, acute hepatitis, fulminant hepatic failure, sepsis, organ transplantation rejection, rheumatic arthritis, cardiac cell apoptosis due to ischemic cardiac diseases, or liver cirrhosis in a patient, which comprises administering a therapeutically effective amount of the compound as defined in claim 1 or pharmaceutically acceptable salt thereof to the patient.

15. A pharmaceutical composition for inhibiting caspase, comprising the compound as defined in claim 6 or pharmaceutically acceptable salt thereof as an active ingredient together with a pharmaceutically acceptable carrier.

16. A use of the compound as defined in claim 6 or pharmaceutically acceptable salt thereof for inhibiting caspase.

17. A method for preventing inflammation and apoptosis in a patient, which comprises administering a therapeutically effective amount of the compound as defined in claim 6 or pharmaceutically acceptable salt thereof to the patient.

18. A method for the treatment or prevention of dementia, cerebral stroke, brain impairment due to AIDS, diabetes, gastric ulcer, cerebral injury by hepatitis, hepatitis-induced hepatic diseases, acute hepatitis, fulminant hepatic failure, sepsis, organ transplantation rejection, rheumatic arthritis, cardiac cell apoptosis due to ischemic cardiac diseases, or liver cirrhosis in a patient, which comprises administering a therapeutically effective amount of the compound as defined in claim 6 or pharmaceutically acceptable salt thereof to the patient.
